SEQUENCE LISTING (8 sequences)

SEQ ID NO: 1 - DNA, 20 bp, Artificial
  primer synthesis
cgtggtgacaagggtgagac

SEQ ID NO: 2 - DNA, 20 bp, Artificial
  primer synthesis
taggtgatgttctgggaggc

SEQ ID NO: 3 - DNA, 24 bp, Artificial
  primer synthesis
gatgcctggaatgccatccccgat

SEQ ID NO: 4 - DNA, 24 bp, Artificial
  primer synthesis
aggcttggtgggattggaggggga

SEQ ID NO: 5 - DNA, 26 bp, Artificial
  primer synthesis
tgaaccggcctttcctgcttctcatg

SEQ ID NO: 6 - DNA, 25 bp, Artificial
  primer synthesis
gcggaagtcaatgtacagctgccgc

SEQ ID NO: 7 - DNA, 25 bp, Artificial
  primer synthesis
caggtcatcacyatyggcaatgagc

SEQ ID NO: 8 - DNA, 25 bp, Artificial
  primer synthesis
cggatgtcmacgtcacacttcatga

(19) United States
(12) Patent Application Publication
(10) Pub. No.: US 2009/0186100 A1
(43) Pub. Date: Jul. 23, 2009

(54) MEDICAMENT FOR ANTI-AGING SKIN CARE AND WOUND HEALING AND USE THEREOF

(75) Inventors: Suying Liu, Taipei Shien (TW); Pei-Fen Yang, Taipei Shien (TW)

Correspondence Address: WPAT, PC;INTELLECTUAL PROPERTY ATTORNEYS, 2030 MAIN STREET, SUITE 1300, IRVINE, CA, 92614, US

(73) Assignee: SAGITTARIUS LIFE SCIENCE CORP., Taipei Shien (TW)

(21) Appl. No.: 12/018,155

(22) Filed: Jan. 22, 2008

(51) Int. Cl. A61K 36/185; A61P 17/02

(52) U.S. Cl. 424/725

(57) ABSTRACT

A composition comprising Semen Lactucae sativae, Fructus oryzae and Resina Boswelliae carterii for prophylaxis or treatment of skin-aging, increasing skin hydration, improving wrinkle; and facilitating wound healing rate and improving wound quality.

### FIELD OF THE INVENTION

The present invention relates to a composition and a method for prophylaxis or treatment of skin-aging; facilitating wound healing rate and improving wound quality.

### BACKGROUND OF THE INVENTION

According to World Health Organization (WHO), in 2025 the population over 60 years old will reach 1 billion, and it will be doubled worldwide by 2050. According to the 2000Census, 12.4% of the US population or 35 million people, were 65 years of age or older. As the baby boomer population reaches the age of 65 beginning in 2011, the numbers will dramatically increase. People 85 years and older are the fastest-growing age groups within the older population. Many intrinsic and extrinsic variables contribute to the aging of society, and improved health care has increased life expectancy. The natural aging process induces inevitable decline of the body organs, including the skin. When a person is in his or her 20's, subtle skin alterations begin, regardless of external environmental exposure. Between 30 and 80 years of age, the epidermis becomes thin due to the decreasing of about 30 to 50% of the turnover rate of the structural components. This decrease compromises nutrient exchanges and communications between the epidermis and dermis, affecting physiological functions including barrier, immunologic, and mechanical functions. The decrease in melanocytes (loss of 10%-20% per decade) and in Langerhans' cells increases the risk of skin cancer and infection.

Skin aging is the result of a combination of chronological (genetic, biological clock), hormonal (estradiol) and environmental (ultraviolet light, smoking, pollution) factors (Guinot C, Malvy Denis J. M, Ambroisine L, Latreille J, Mauger E, Tenenhaus M, Morizot F, Lopez S, Le Fur I, Tschachler E. Relative contribution of intrinsic versus extrinsic factors to skin aging as determined by a validated skin age score. Archives of Dermatology. 138(11): 1454-1460, 2002; Son E D, Lee J Y, Lee Serah, Kim M S, Lee B G, Chang I S, Chung J H. Topical application of 17[beta]-Estradiol increases extracellular matrix protein synthesis by stimulating TGF-[beta] signaling in aged human skin in vivo. J. Investigative Dermatology. 124(6): 1149-1161, 2005). Aged skin often has reduced numbers of keratinocytes and fibroblasts, and in the amount and organization of connective tissue, which causes the interface between the epidermis and the dermis to flatten and thin (There is a loss of dermal thickness in 20% of the elderly as the dermis becomes avascular). Degradation and deregulation of collagen and collagen synthesis impair the tensile strength and the ability to heal skin properly. There is also a progressive loss of elastic recovery and hydration of the skin, and the aging appearance becomes more prevalent (Varani J., Dame M K, Rittie L, Fligiel S E G, Kang S, Fisher G J, Voorhees J J. Decreased collagen production in chronologically aged skin. Am J Pathol 168(6): 1861-1868, 2006).

Skin connective tissue is comprised primarily of fibrillar collagen bundles and elastic fibers that give strength and resilience to skin, and glycosaminoglycans that retain water to give skin radiance and cushion. Their degeneration with aging causes skin to become rigid, fragile, wrinkled, dry and aged in appearance and easily injured by the environment. The alteration of subcutaneous fat distribution can affect the main functions of the subcutaneous layer of skin. Although the proportion of subcutaneous fat actually increases until age 70, the overall volume of fat diminishes with age. Thinning of subcutaneous tissue in the face, hands, shins, and feet causes the skin to become dry, flaky, and itchy and decreases insulation, cushion, and protection against mechanical injury. Approximately 3.5% of the geriatric population suffers from leg ulcers with a recurrent rate as high as 70%.

The increased prevalence of wounds in the elderly may be caused by immobility, disease, or the changes in the skin that lead to poor response to stress and wound healing. Cutaneous wound healing is a complex process encompassing a number of overlapping events including an inflammation phase (immune cell recruitment, cytokine release), proliferation phase (cell proliferation, matrix deposition, re-epithelialization, granulation) and maturation phase (resolution of inflammation, formation of a mature scar and remodeling). Morbidity associated with age-related delayed wound healing has an enormous social and financial impact; the projected increase in the elderly population will further exacerbate this problem.

### BRIEF DESCRIPTION OF DRAWINGS

FIG. 1 illustrates effect of SS304L on the deposition of collagen and glycosaminoglycan in dermal fibroblast treated with SS304L for 1 week. Lane 1 and 2: Safranin O stain for GAG, Lane 3 and 5: Sirius red stain for collagen. A-1, B-1, A-3, B-3: SS304L at 0.01 mg/ml; A-2, B-2, A-4, B-4: SS304L at 0.03 mg/ml; E-2, F-2, E-4, F-4: control.

FIG. 2 illustrates effect of SS304L on gene expressions in human dermal fibroblasts treated with SS304L at 0.01 mg/ml for 1 week. The cell layers were washed with PBS and lysed. The RT/PCR was performed to assess the expression of type 1 collagen, MMP-2 and TGF-β. The quantities of expressed fragments were adjusted by expressed house keeping β-actin, and analyzed by NIH Image J. From left to right: molecular weight ladder, control, SS304L.

FIG. 3 illustrates effect of SS304L on MMP-2 activity in human dermal fibroblasts treated with SS304L at 0.01 mg/ml for 1 week. The cell layers were washed with PBS and serum free media were added and cultured for a further 24 hours. The gelatin zymography was performed to assess the activity of MMP secreted. The relative quantities of digested clear bands were analyzed by NIH Image J (SS: SS304L).

FIG. 4 illustrates effect of SS304L on skin hydration measured by CM825 in women treated with SS304L for various time intervals.

### SUMMARY OF THE INVENTION

The present invention provides a composition, SS304L, comprising Semen Lactucae sativae, Fructus oryzae and Resina Boswelliae carterii.

The present invention also provides a method of preparing the composition of the present invention.

The present invention further provides a method for prophylaxis or treatment of skin-aging of a subject comprising administrating an effective dose of the composition of the present invention to a subject.

The present invention further provides a method for facilitating wound healing rate and improving wound quality of a patient comprising administrating an effective dose of the composition of the present invention to a patient.

### DETAILED DESCRIPTION OF THE INVENTION

The preliminary views disclose loss of skin collagen and glycosaminoglycan (GAG) during aging results from increased matrix metalloproteinase degradation and reduced collagen production contributing to wrinkle formation and the decreased elasticity of aging skin. High-molecular-weight linear GAG is well known for its ability to retain water and maintain the extracellular matrix. The mechanism responsible for these alterations involves reduction of transforming growth factor-β (TGF-β), which is the most potent regulator of collagen production and GAG synthesis. TGF-β is a multifunctional cytokine that plays an important role in regulating cell growth, differentiation, and the biosynthesis of extracellular mtrix (Massagué J. TGF-[beta] signal transduction. Annu. Rev. Biochem. 67: 753-791, 1998; Massagué J. How cells read TGF-[beta] signals. Nat. Rev. Mol. Cell Biol. 1:169-178, 2000). TGF-β-1 up-regulates GAG synthesis in fibroblasts by inducing the expression of HAS2 mRNA (Yoshida H, Sayo T, Kusaka A, Furukawa M, Okui M*, Komaki R*, Inoue S. (−)-Muscone and Saffron Extract Selectively Regulate Hyaluronan Synthesis at the MRNA Level in Human Skin Cells. [34th] Annual European Society for Dermatological Research (ESDR) Meeting: POSTERS: Bullous Diseases, Extracellular Matrix & Genetics: Extracellular matrix/Connective Tissue. Journal of Investigative Dermatology. 123(2): A17, 2004). Changes in TGF-beta₁ temporally correlate with the development of healed wounds, suggesting that TGF-beta₁ may play a central role in wound repair. TGF-beta₁ expression has been identified in granulation tissue and in the upper layers of healing epidermis during the repair process. A strong up-regulation of TGF-beta₁ gene and protein expression after injury has been demonstrated (Wang X, Polo M, Ko F, Robson M C. Endogenous TGF-Beta1 Gene and Protein Changes During Human Partial Thickness Wound Healing. Wounds 10(3): 76-82, 1998).

One factor contributing to non-healing chronic wound of aging may be inhibition of fibroblast proliferation and induction of a stress-induced premature senescence by the continuing inflammation found in chronic wounds. Senescent fibroblasts exhibit an extracellular matrix degradative phenotype that contributes to non-healing. Accumulation of greater than 15% senescent fibroblasts has been described as a threshold beyond which wounds become difficult to heal. The ratio of senescent to non-senescent cells is therefore critical to determine the response to treatment, and adjunctive therapies that modulate this ratio in favor of non-senescent cells are likely to enhance therapeutic healing rates (Harding K G, Moore K, Phillips T J. Wound chronicity and fibroblast senescence—implications for treatment. Int Wound J. 2(4): 364-368, 2005).

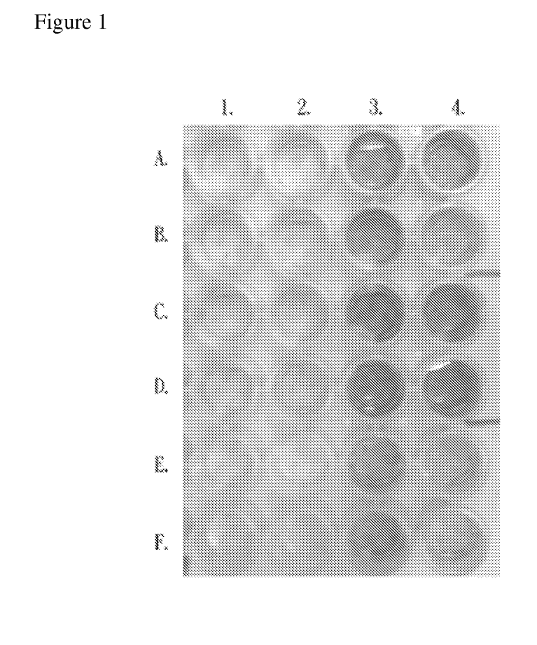

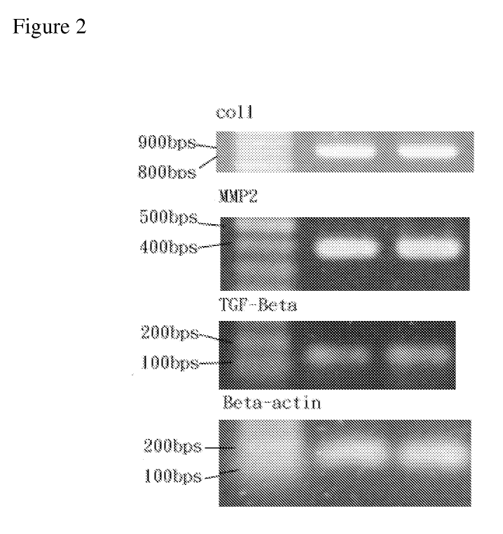

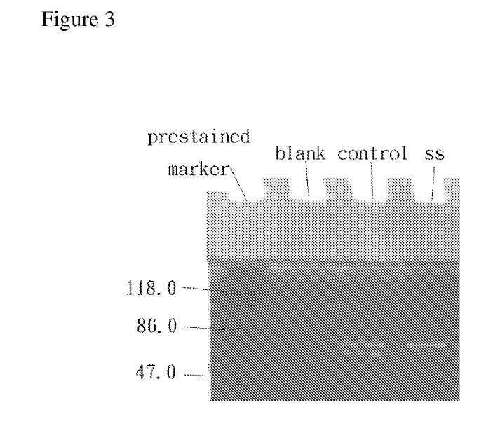

The composition (SS304L) increased the proliferation of dermal fibroblasts by 20% at 0.2 mg/ml (Table 1) that overcome the threshold of 15% senescent fibroblasts in non-healing wounds. SS304L increased deposition of collagen and GAG dose dependently (Table 3, 4). SS304L did not affect the expressions of type I collagen and MMP-2, however, it stimulated TGF-β expression after 1 week of treatment (FIG. 2, Table 5). The increased TGF-β by SS304L appeared to mediate the increases of collagen and GAG depositions (Eickelberg O, Kohler E, Reichenberger F, Bertschin S, Woodtli T, Erne P, Perruchoud Andre P, Roth M. Extracellular matrix deposition by primary human lung fibroblasts in response to TGF-beta1 and TGF-beta3. Am. J. Physiol. 276:814-824, 1999). SS304L also inhibited the active form of MMP-2 (FIG. 3) to ensure the increased deposition of collagen (FIG. 1, Table 3).

Reactive oxygen species (ROS) generated by UV irradiation play an important role in UV-induced skin damages. Chronic exposure of dorsal skin of hairless mouse to UV induced wrinkle formation and the gelatinase activities of MMP-9 and MMP-2 (Park C, Lee M, Kim K, Cho K, Eun H, Yoo I, Chung J. Prevention of ultraviolet radiation-induced premature skin aging in hairless mice by the novel compound, Melanocin A. Photochemistry and photobiology, 82(2): 574-578, 2006). Anti-oxidants are able to block ROS, thus inhibit gelatinase activity to prevent UV induced skin aging. SSL304L showed SOD mimetic anti-oxidative activity (Table 7) that had effect on inhibiting gelatinase activity and degradation of collagen.

It is well documented that aging impairs wound healing (Gosain A, DiPietro L A. Aging and wound healing. World J Surg 28(3): 321-326, 2004; Ashcroft G S, Horan M A, Ferguson M, W. J. Aging is associated with reduced deposition of specific extracellular matrix components, an upregulation of angiogenesis, and an altered inflammatory response in a murine incisional wound healing model. Journal of Investigative Dermatology 108: 430-437, 1997; Ashcroft G S, Mills S J, Ashworth J J. Aging and wound healing Biogerontology 3(6): 337-345, 2002), with an excessive local inflammation, an increased proteolytic activity, a decrease in fibronectin in the wounds, an over the threshold of fibroblast senescence, a delay in re-epithelialization and appearance of extracellular matrix components, and an overall decrease in collagen I and III deposition in the wounds in elderly.

In an aging mouse, full thickness excision wounds, on day 3 post wounding, 50% of the wounds treated with vehicle control showed significant inflammation, while the arginine and SS304L treated wounds showed no apparent sign of inflammation. On day 7, the re-epithelization/granulation process was significantly facilitated by arginine and SS304L. On day 10, the 1% arginine, 0.1% SS304L and 1% SS304L treated wounds showed about ⅓ to ½ unhealed wound areas compared to the areas of the vehicle treated wounds. SS304L showed dose-dependent healing effect. By day 14, the drug treated wounds appeared to be completely healed by arginine and SS304L treatments, while vehicle treated wounds still were not completely healed. By day 18, the remodeling/maturation process seemed to have proceeded better by the SS304L treatments with normal appearance to hair growth and no sign of significant scar formation; conversely, in vehicle-control wounds, 6/8 had some fibrotic tissue formed. L-arginine has been shown to enhance wound strength and collagen synthesis in rodents and humans. Its supplementation enhances wound healing following trauma/hemorrhagic shock. (Shi H P, Wang S M, Zhang G X, Zhang Y J, Barbul A. Supplemental L-arginine enhances wound healing following trauma/hemorrhagic shock. Wound Repair Regen. 15(1): 66-70, 2007). Arginine also significantly enhanced wound healing, and reduced hospital stay days in severe trauma patients (Peng X, Yi D, Fan S Z, Liao Z J, Yao Y Z, Cong T Q, Xiang J, Wang Z Y, Meng D S, You Z Y. Analysis of the influence on the prognosis and safety of arginine in patients with severe trauma and burns—a multi-center randomized double blinded, placebo controlled clinical trail in 86 patients Zhonghua Shao Shang Za Zhi. 22(4): 243-6, 2006). Local application of L-arginine promotes anal fissure healing by reducing anal resting pressure and improving anodermal blood flow (Gosselink M P, Darby M, Zimmerman D D, Gruss H J, Schouten W R. Treatment of chronic anal fissure by application of L-arginine gel: a phase II study in 15 patients. Dis Colon Rectum. 48(4): 832-7, 2005). 0.1% SS304L showed comparable effect with 1% arginine on wound healing in aging mice.

Two of the most important pathways to inflammation are mediated by the enzymes 5-lipoxygenase (5-LO) and cyclooxygenase (COX). These are parallel pathways that result in the generation of leukotrienes and prostaglandins, respectively, which play important roles in the initiation and progression of the inflammatory response. These vasoactive chemotaxins promote infiltration of inflammatory cells into tissues and serve to prolong the inflammatory response, which is crucial in would healing. Cyclooxygenase-2 is up-regulated shortly after dermal injury and it has been shown to have important activity during the repair process. Its main product in the skin, prostaglandin E2 (PGE2), modulates both inflammatory and fibrotic processes and outcome during wound healing (Sandulache V C, Parekh A, Li-Korotky H-S, Dohar J E, Hebda P A. Prostaglandin E2 differentially modulates human fetal and adult dermal fibroblast migration and contraction: implication for wound healing. Wound Repair and Regeneration. 14(5): 633-643, 2006). In early fetal skin, wounds exhibit a unique pattern of healing leading to regeneration with little or no inflammation, faster re-epithelialization, and no scarring (Liu W, Cao Y. Longker M T. Gene therapy of scarring: a lesson learned from fetal scarless wound healing. Yonsei Med J. 2001; 42(6): 634-645). In contrast to fetal wound healing, adult dermal wound healing results in imperfect repair and scar formation. Fibroblasts are responsible for the contraction and remodeling of the wound matrix, which is influenced by inflammatory mediators including PGE2 (Parekh A, Sandulache V C, Lieb A S, Dohar J E, Hebda P A. Differential regulation of free-floating collagen gel contraction by human fetal and adult dermal fibroblasts in response to prostaglandin E2 mediated by an EP2/cAMP-dependent mechanism. Wound Repair Regen. 15(3): 390-398, 2007). 5-lipoxygenase and leukotriene receptors are present in post surgical peritoneal wound and incision wound (Chegini N, Rossi M J, Holmdahl L. Cellular distribution of 5-lipoxygenase and leukotriene receptors in post surgical peritoneal wound repair. Wound Repair Regen. 5(3): 235-242, 1997), that also play important role in healing. The process of inflammation with the rush of chemicals and cells to the area of injury serves to fight infection and stimulates the processes of re-epithelialization and remodeling, it also has destructive consequences that are detrimental. The un-controlled lysosomal enzyme release can lead to the digestion of normal tissues; inflammatory tissue edema and tissue ischemia are also painful and difficult to resolve.

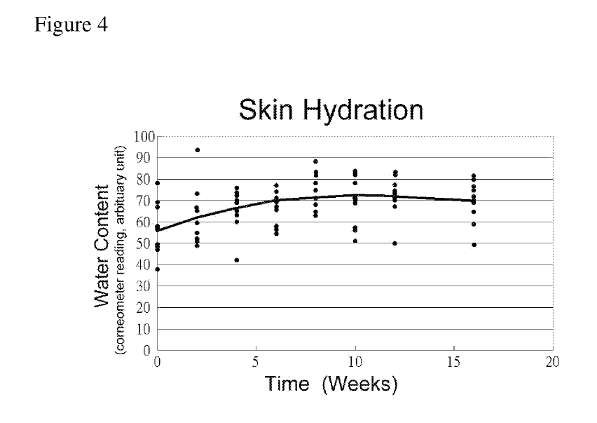

The composition (SS304L) had COXII and 5-LO dual inhibitory activities with IC₅₀ of 10 μg/ml. Therefore, SS304L was able to decrease inflammation associated with aging and environmental insults such as UV, trauma, and wounding. The facilitating healing effects of SS304L on full thickness excision wounds of aging mice demonstrated by keeping inflammation in check (COXII, 5-LO inhibition), earlier proliferation/re-epithelization/granulation (fibroblast proliferation, collagen and GAG deposition via induction and modulation of endogenous TGF-β expression), and better remodeling/maturation (SOD mimetic anti-oxidation activity, MMP activity modulation) resulted in regeneration/repair of skin wound to ½ to ⅓ of the time required with less inflammation and fibrotic scar formation. Clinically, treatment of SS304L for three months showed significant improvement of skin aging condition and hydration as assessed by dermatologists (Table 10) and CK Corneometer measurement (FIG. 4). Subjective assessment of participants was also significantly improved globally (Table 10). Objective Corneometer measurement demonstrated that the skin hydration increased 26.9% (p<0.05) after three months of treatment, and the effect lasted at least one month after discontinuation of dosing (increased 24.0%, p<0.05) (Table 11, 12) without apparent side effects (Table 13).

In the present invention, SS304L is disclosed to stimulate the proliferation of dermal fibroblasts, increase the deposition of collagen and glycosaminoglycan, increase the expression of TGF-β, inhibit the active form of MMP-2, show SOD mimetic anti-oxidation activity, and show anti-COX II and 5-LO activity. This wide spectrum of anti-aging, anti-inflammatory and regenerative/repair activity is evident by (1) facilitating the wound healing rate to ½ to ⅓ of the time required and improving quality of wound with no evident fibrotic tissue formed by modulating inflammation, re-epithelization, maturation and remodeling of the wound bed in mice and (3) increasing the skin hydration by 26.9% after three months of treatment, and 24.0% one month post treatment with global improvements on skin aging conditions in women age 40 to 50 years old.

Accordingly, the present invention provides a composition (SS304L) comprising Semen Lactucae sativae, Fructus oryzae and Resina Boswelliae carterii.

In a preferred embodiment of the present invention, the weight to weight ratio of Semen Lactucae sativae, Fructus oryzae and Resina Boswelliae carterii is 2-20: 1-5: 0.1-1. In more preferred embodiment of the present invention, the weight to weight ratio is 5:2:0.5.

According to the present invention, Resina Boswelliae carterii can be resin containing Resina Boswelliae carterii.

In addition, the composition of the present invention can be in a form of, but not limited, tablet, film coated tablet, capsules, soft gel capsules, granules, powder, pearl, liquid, emulsion, injection solution, ointment, lotion, cream, serum, spray, inhaler, skin covering or wound dressing by employing with a pharmaceutically acceptable excipient, carrier or diluent.

The present invention also provides a method of preparing the composition comprising: (1) extracting Semen Lactucae sativae, Fructus oryzae, Resina Boswelliae carterii with 5 to 10 fold volume of water at 95 to 105° C. for two hours twice, and (2) mixing the effective amount of the extract with a pharmaceutically acceptable carrier, excipient or diluent to make into the expecting dosage forms.

The present invention further provides a method for prophylaxis or treatment of skin-aging of a subject comprising administrating an effective dose of the composition of the present invention to a subject. The weight to weight ratio of the composition is 2-20:1-5:0.1-1. The more preferable ratio is 5:2:0.5. The composition is administered via oral, intramuscular injection, subcutaneous injection, intra-venous injection, mucosal membrane or topical routes in appropriate dosage forms.

The present invention further provides a method for facilitating wound healing rate and improving wound quality of a patient comprising administrating an effective dose of the composition of the present invention to a patient. The weight to weight ratio of the composition is 2-20:1-5:0.1-1. The more preferable ratio is 5:2:0.5. The composition is administered via oral, intra-muscular injection, subcutaneous injection, intra-venous injection, mucosal membrane or topical routes in appropriate dosage forms.

### EXAMPLES

The examples below are non-limiting and are merely representatives of various aspects and features of the present invention.

Cell Sample

Human dermal fibroblasts (HFB) were cultured from foreskin with consent by cell emigration from ˜1 mm³ skin pieces in DMEM+1 mM sodium pyruvate+2 mM glutamine+100 U/ml penicillin+100 ug/ml streptomycin+10% fetal calf serum (FCS) (C-DMEM) under humidified atmosphere containing 5% CO₂ at 37° C.

Reagents

Chemicals were from Sigma unless indicated otherwise. Tissue culture media and reagents were from Gibco. The culture plastic wears were from Nunc. SS304L: The herbal composition of the present invention

### Example 1

### HFB Proliferation

1×10⁴ HFB were seeded in a 96 multi-well culture dish. 48 hours later, the cells were treated with various concentrations of SS304L in C-DMEM under humidified atmosphere containing 5% CO₂ at 37° C. The cells were exposed to drugs for 72 hours, and then treated with 0.5 mg/ml MTT for 4 hours at 37° C. The reduced formazan was dissolved in isopropanol containing 0.04 N HCl, and measured at A570 nm by an ELISA reader (Molecular Device).

Results:

Stimulation of HFB Proliferation

TABLE 1

| SS304L | | | Vitamin A | | | Vitamin E | | | Estradiol | | |
|---|---|---|---|---|---|---|---|---|---|---|---|
| Conc. | Mean | Inc. | Conc. | Mean | Inc. | Conc. | Mean | Inc. | Conc. | Mean | Inc. |
| (mg/ml) | OD | (%) | (uM) | OD | (%) | (uM) | OD | (%.) | (nM) | OD | (%.) |
| 0.2 | 0.159 | 20 | 10 | 0.129 | −6 | 300 | 0.120 | −16 | 100 | 0.131 | −4 |
| 0.07 | 0.144 | 5 | 3 | 0.124 | −12 | 100 | 0.126 | −9 | 30 | 0.132 | −3 |
| 0.02 | 0.134 | −5 | 1 | 0.131 | −4 | 30 | 0.131 | −4 | 10 | 0.137 | 2 |
| 0.007 | 0.139 | 0 | 0.3 | 0.131 | −4 | 10 | 0.144 | 9 | 3 | 0.139 | 4 |
| 0.002 | 0.134 | −5 | 0.1 | 0.128 | −7 | 3 | 0.135 | 0 | 1 | 0.131 | −4 |
| 0 | 0.139 | | 0 | 0.135 | | 0 | 0.135 | | 0 | 0.135 | |
| Blank | 0.040 | | Blank | 0.040 | | Blank | 0.040 | | Blank | 0.040 | |

As shown in Table 1, at 0.2 mg/ml, SS304L stimulated HFB proliferation 20% after treatment with SS304L for 3 days. Under the same experimental conditions, vitamin A, E or estradiol did not stimulate HFB proliferation.

### Example 2

### Collagen and Glycosaminoglycan (GAG) Detection and Measurement

5×10⁴ HFB were seeded in a 24 or 48 multi-well culture dish. After reaching confluency, the cells were treated with various concentrations of SS304L in C-DMEM+50 μg/ml vitamin C under humidified atmosphere containing 5% CO₂ at 37° C. The cells were exposed to drugs for 1 week, and the cells were either fixed in 10% buffered formalin and stained with Sirius red or Safranin O, or measured by papain/1,9-dimethyl methylene blue (Enobakhare, B. O., Bader, D. L., Lee, D. A., Quantification of Sulphated Glycosaminoglycans in Chondrocyte/Alginate Cultures, by use of 1,9-Dimethylmethylene blue. Analytical Biochemistry 243: 189-191, 1996) for GAG or Sirius red elution for collagen (Koslowski R, Knoch K, Kuhlisch E, Seidel D, Kasper M. Cathepsins in bleomycin-induced lung injury in rat. Eur. Respir J. 22(3): 427-35, 2003).

Results:

Increase of Extracellular Collagen and Glycosaminoglycan

As shown in FIG. 1, the deposition of extracellular collagen and glycosaminoglycan were increased by treatment of HFB with SS304L for 1 week.

### Example 3

### Increase in Quantity of Dermal Collagen

Dermal fibroblasts were treated with SS304L for 1 week. The cells were fixed, stained with Sirius red and eluted with 0.1 N NaOH/Methanol. The concentration of collagen was obtained by comparison with a standard curve of spotted acid soluble type I collagen measured at A540.

Results:

Quantities of increase in extracellular collagen by SS304L

TABLE 2

| | Concentration | Collagen | |
|---|---|---|---|
| | (mg/ml) | (ug/ml) | Increase (%) |
| Control | | 52.51 | |
| SS304L | 0.01 | 55.41 | 5.5 |
| SS304L | 0.05 | 58.87 | 12.1 |

As shown in Table 2, one week of treatment of HFB by SS304L at 0.01 and 0.05 mg/ml increased collagen by 5.5% and 12.1% respectively.

### Example 4

### Increase in Quantity of Dermal GAG

Dermal fibroblasts were treated with SS304L for 1 week. The cell layers were digested with papain and the released GAG was measured by dimethyl methylene blue at A525 compared with a chondroitin sulfate standard curve.

Results:

Quantities of increase in extracellular GAG by SS304L

TABLE 3

| | Concentration | | |
|---|---|---|---|
| | (mg/ml) | GAG (ug/ml) | Increase (%) |
| Control | | 6.37 | |
| SS304L | 0.01 | 8.57 | 34.5 |
| SS304L | 0.05 | 12.18 | 91.1 |

As shown in Table 3, one week of treatment of HFB by SS304L at 0.01 and 0.05 mg/ml increased GAG by 34.5% and 91.1% respectively.

### Example 5

### RT/PCR

The RNA of cells treated with the present invention for 1 week was extracted with Tri-Reagent (Sigma). 800 ng of total RNA was reverse transcribed by Omniscript reverse transcriptase (Qiagen, Valencia, Calif.) in buffer containing 1 μM random primer, 0.5 mM each of dNTP, and 10 units of ribonuclease inhibitor. Reverse transcription was carried out at 37° C. for one hour by a programmable thermal controller (PTC-100, MJ Research, Inc., Watertown, Mass.). The reaction was terminated by raising the temperature to 93° C. for five minutes.

Polymerase chain reaction (PCR) amplification was performed with primer pairs as listed in Table 4. PCR was performed in a thermal controller (PTC-100, MJ Research, Inc., Watertown, Mass.) with master mix (HotStarTaq DNA polymerase, Qiagen) at initial denaturation at 95° C. for 15 minutes, followed by 25 cycles of 94° C. for 1 minute, 58° C. for 40 seconds, and 72° C. for 40 seconds. The final cycle extended 72° C. for 10 minutes. The PCR products accompanied by 100 bp ET marker were separated by 1.2% agarose gel, stained with ethidium bromide and visualized under UV light. A Polaroid picture was taken by Foto/Phoresis UV documentation system (Fotodyne Inc.). Beta-actin expression under the same experimental conditions was used as a reference to normalize the expressions. The relative quantities of expressions were analyzed by Image J.

TABLE 4

| | |
|---|---|
| Gene | Primer Pair |

| | |
|---|---|
| COL 1 | F: CGTGGTGACAAGGGTGAGAC |
| | R: TAGGTGATGTTCTGGGAGGC |
| MMP-2 | F: GATGCCTGGAATGCCATCCCCGAT |
| | R: AGGCTTGGTGGGATTGGAGGGGGA |
| TGF-β | F: TGAACCGGCCTTTCCTGCTTCTCATG |
| | R: GCGGAAGTCAATGTACAGCTGCCGC |
| β-Actin | F: CAGGTCATCACYATYGGCAATGAGC |
| | R: CGGATGTCMACGTCACACTTCATGA |

Results:

Increase in expression of TGF-β by SS304L

TABLE 5

| Gene | Ratio of SS304L:Control |
|---|---|
| Col I | 1.03:1 |
| MMP-2 | 1.02:1 |
| TGF-β | 1.37:1 |

As shown in FIG. 2 and Table 5, the expression of Col I and MMP-2 were not affected by treatment of SS304L. However, the expression of TGF-β was increased 37% by SS304L treated for 1 week.

### Example 6

### Matrix Metalloproteinase (MMP) Zymography

The 1 week-drug treated HFB were washed with PBS, and serum free DMEM were added and collected 24 hours later for MMP analysis by zymography. The protein concentration of culture supernatants was determined by Bradford protein assay (Bio Rad). Equal amount of protein of each culture medium was mixed with equal volume of 2×non-reducing SDS sample buffer, and incubated at room temperature for two hours for sample to denature. The samples were loaded into each well of a 10% SDS polyacrylamide gel containing 1 mg/ml of bovine gelatin (Sigma), and run at 20 mA in ice bath by a BioRad Mini Protein gel apparatus. After dye front reached the bottom of the gel, the gel was washed three times, 15 minutes each, with 2.5% Triton X-100. Then, the gel was renatured in developing buffer (50 mM Tris-HCl, pH7.6, 10 mM CaCl₂, 50 mM NaCl, 0.05% Brij35) at 37° C. for 24 hours. The gel was stained with 0.1% Coomassie blue (Sigma) in 40% methanol and 10% acetic acid, and destained in 30% methanol and 7% acetic acid. The enzyme digested clear bands were visualized in the blue background, and analyzed by Image J.

Results:

Effect of SS304L on MMP-2 by zymography

TABLE 6

| MMP-2 | Ratio of SS304L:Control |
|---|---|
| Latent form (72K) | 0.97:1 |
| Active form (68K) | 0.59:1 |

As shown in FIG. 3 and Table 6, the activity of latent form of MMP-2 was not affected by SS304L, however, the activity of active form was decreased to 59% of the level of the control.

### Example 7

### Free Radical Scavenger, SOD Mimetic Activity

SS304L or vehicle was incubated with 0.12 mM xanthine, 6 mU xanthine oxidase, 27 μM nitroblue tetrazolium (NBT), 0.11 mM EDTA, 0.005% bovine serum albumin and Na₂CO₃ at pH 10.5 at 25° C. for 20 minutes. Conversion of xanthine to uric acid +O⁻+NBT to formazan was then determined by measurement of absorbance at 595 nm and percent inhibition by superoxide dismutase or SS304L was calculated.

Results:

TABLE 7

| Treatment | Concentration | | Inhibition (%) |
|---|---|---|---|
| (−)-Epicatechin gallate | 6.1 | μM | 50 |
| *Superoxide dismutase (SOD) | 0.0084 | μM | 50 |
| SS304L | 100 | μg/ml | 50 |

*indicates standard reference agent used

As shown in Table 7, SS304L demonstrated 50% SOD mimetic activity at 100 μg/ml.

### Example 8

### Inhibition of Cyclooxygenase II (COX II)

Human recombinant COX II expressed in Sf21 cells (Sigma, C-0858) was used. SS304L or vehicle was reinsulated with 0.11 U enzyme, 1 mM reduced glutathione, 500 μM phenol and 1 μM hematin in Tris-HCl, pH 7.7 at 37° C. for 15 minutes. 0.3 μM arachidonic acid as substrate was added and the reaction was terminated by addition of 1 N HCl after 5 minutes. The converted PGE₂ was measured by an Amersham EIA kit following centrifugation. SS304L was assayed at 10 and 100 μg/ml.

Results:

TABLE 8

| Treatment | Concentration | | Inhibition (%) |
|---|---|---|---|
| SS304L | 10 | μg/ml | 42 |
| SS304L | 100 | μg/ml | 97 |
| Nimesulide | 1 | μM | 50 |

As shown in Table 8, IC₅₀ for SS304L was about 10 μg/ml. The IC₅₀ for standard COX II inhibitor, Nimesulide, was 1 μM.

### Example 9

### Inhibition of Lipoxygenase 5 (5-LO)

Human peripheral blood mononuclear leukocytes (PBML) were isolated by Ficoll-Paque density gradient centrifugation. SS304L or vehicle was reinsulated with PBML (5×10⁶ cells/ml) in HBSS buffer, pH 7.4 at 37° C. for 15 minutes. After incubation with 30 μM A 23187 for 15 minutes, the reaction was terminated by addition of 1 N HCl. Following neutralization with NaOH and centrifugation, LTB₄ in the supernatant was measured using an EIA kit. SS304L was assayed at 10, and 100 μg/ml.

Results:

TABLE 9

| Treatment | Concentration | | Inhibition (%) |
|---|---|---|---|
| SS304L | 10 | μg/ml | 12 |
| SS304L | 100 | μg/ml | 90 |
| NDGA | 0.13 | μM | 50 |

NDGA = Nordihydroguaretic acid

As shown in Table 9, IC₅₀ for SS304L was between 10 and 100 μg/ml. The IC₅₀ for standard 5-LO inhibitor, Nordihydroguaretic acid, was 0.13 μM.

### Example 10

### Full Thickness Excision Wound Healing in Mice

Aging male ICR mice at body weights of 40 to 50 grams (Age: 14 to 16 weeks) were lightly anesthetized with ether. 0.7 mm in diameter full thickness excisions were conducted at the dorsal back skin after shaving and 70% ethanol sterilization. Immediately after excision, the wounds were covered with either vehicle (sterile 1% carboxymethyl cellulose, 25 μl) at first wound site of all mice, and with 1% arginine, 0.1% SS304L, or 1% SS304L at the second wound site. Each group contained 4 to 6 mice. The drugs were applied on the day of photography (Day 0, 1, 2, 3, 4, 7, 10, 14, 18). The areas of the wounds and the rates and qualities of healing were compared at each time point. Arginine was used as comparison due to the similarity of part of its action mechanisms to SS304L.

Results:

Comparing the sizes and morphology of the wounds in the photographs in respect to a reference ruler, on day 3, 50% of the wounds treated with vehicle control showed inflammation, while the arginine and SS304L treated wounds showed no apparent sign of inflammation. On day 7, the re-epithelization/granulation process was significantly facilitated by arginine and SS304L. On day 10, the 1% arginine, 0.1% SS304L and 1% SS304L treated wounds showed about 30% to 50% unhealed areas compared to the wound areas of those of the vehicle treated controls. SS304L showed dose-dependent effect. By day 14, the drug treated wounds appeared to be completely healed by arginine and SS304L treatments, while vehicle treated wounds still were not completely healed. By day 18, the remodeling/maturation process seemed to have proceeded better by the SS304L treatments with normal appearance of hair growth and no sign of significant scar formation; on the contrary, in vehicle-control wounds, 6/8 had some fibrotic tissue formed.

In summary, SS304L at 0.1 and 1% facilitated the healing rates dose dependently to about ½ to ⅓ of the time required for healing in normal aged mice. SS304L at 0.1% showed the same effect as arginine at 1%. SS304L also improved the quality of the healed wounds with less fibrotic tissue formations.

### Example 11

### Clinical Study

10 female volunteers who were between 40 and 50 years of age with an overall aging skin score of 4-9 in the forehead, eye and lip areas, were recruited and underwent a total of 12 weeks treatment. Volunteers took SS304T twice daily during the study month 1, 2 and 3. And then a post follow up visit after 1 month without any treatment was scheduled. During treatment period, subjects were instructed to follow a comprehensive skin care and sun avoidance program. (A) Compliance, elasticity, stiffness, skin color, skin tones/wrinkles, histology and (B) skin hydration in combination of objective and subjective assessments were analyzed. (C) Blood and urine were collected and analyzed at baseline and 12 weeks for evaluation of safety.

Clinical Assessments: The investigator evaluated the skin of the forehead, eye and lip areas for sagging, puffiness, wrinkles, overall aging skin on a scale of 0 (none) to 9; The overall appearance of aging skin of the entire face with respect to wrinkles, puffiness and sagging on a scale of 0 (none) to 9.

Global Improvement Scores: The investigator evaluated the global improvement of the subject facial appearance as compared to a baseline photograph and scored such improvement as: −1=worse, 0=no change, 1=slightly improved, 2=improved, 3=much improved.

The subject's self-evaluated improvement in their own skin condition by drawing a vertical line through a 162 mm horizontal visual assessment scale (VAS) with the left end labeled with a 1 and the right end with a 10. The parameters evaluated were as follows: Skin lifted to more sagging; Skin less/smoother wrinkles to more/deeper wrinkles. Entire face for overall appearance of aging skin by much improved/looks younger to worse/more aging.

At each visit, the hydration status of the skin of the cheek area was assessed by calibrated Comeometer (CM 825, Courage and Khjazaka, Koln, Germany). Three areas in the cheek were measured, and average recorded. The value was compared to the baseline for objective assessment.

Results:

(A) Subjective assessments by investigator and volunteers

TABLE 10

| | Week | Sum of Square | SD | P | |
|---|---|---|---|---|---|
| Investigator's evaluation | | | | | |
| Wrinkle around the eyes: | 12 | 1.45 | 0.46 | 0.003 | ** |
| | 16 | 1.01 | 0.08 | <0.001 | ** |
| Wrinkle around the mouth: | 12 | 1.57 | 0.41 | <0.001 | ** |
| | 16 | 0.95 | 0.07 | <0.001 | ** |
| Wrinkle in the forehead: | 12 | 1.10 | 0.47 | 0.023 | * |
| | 16 | 1.02 | 0.08 | <0.001 | ** |
| Hydration of the cheek | 12 | 1.93 | 0.51 | <0.001 | ** |
| | 16 | 0.89 | 0.09 | <0.001 | ** |
| Elasticity of the cheek: | 12 | 1.33 | 0.62 | 0.068 | |
| | 16 | 0.98 | 0.71 | 0.092 | |
| Pigmentation of the cheek: | 12 | 0.95 | 0.61 | 0.125 | |
| | 16 | 1.04 | 0.85 | 0.112 | |
| Global assessment: | 12 | 1.08 | 0.30 | <0.001 | ** |
| | 16 | 1.00 | 0.05 | <0.001 | ** |
| Self evaluation | | | | | |
| Sagging of the face: | 0 | 5.70 | 2.11 | | |
| | 12 | 5.50 | 2.07 | 0.705 | |
| | 16 | 5.50 | 2.01 | 0.726 | |
| Wrinkle in the forehead: | 0 | 6.00 | 3.06 | | |
| | 12 | 5.50 | 1.84 | 0.537 | |
| | 16 | 5.50 | 1.78 | 0.581 | |
| Global assessment: | 0 | 7.20 | 1.87 | | a |
| | 12 | 6.10 | 1.91 | 0.017 | b |
| | 16 | 6.30 | 2.06 | 0.054 | ab |

(B) Objective assessment of skin hydration by CK Corneometer CM825

Analysis of repeated measurement data was used to compare differences between base line (0 week), three months treatment (12 weeks) and one month's evacuation (16 weeks) in each item. A p value of <0.05 was considered significant for all tests. There was no statistic difference between the groups with the same symbol (a, b).

As shown in Table 10, investigator's evaluations of wrinkle around the eyes, mouth, forehead was significantly reduced after 3 months treatment, and the effect lasted at least 1 month post-treatment. The hydration of the cheek was significantly increased after 3 months treatment, and the effect lasted for at least 1 month post-treatment. Global assessment was also significantly improved after 3 months treatment, and the effect lasted for 1 month after discontinuation of treatment. Self-global assessment was significantly improved after 3 months treatment

TABLE 11

| week | average | p | |
|---|---|---|---|
| 0 | 56.28 ± 11.23 | | |
| 2 | 61.48 ± 12.84 | 0.110 | |
| 4 | 65.23 ± 9.33 | 0.021 | * |
| 6 | 65.66 ± 7.63 | 0.032 | * |
| 8 | 73.73 ± 8.13 | 0.001 | ** |
| 10 | 69.82 ± 11.16 | 0.010 | ** |
| 12 | 71.43 ± 8.98 | 0.003 | ** |
| 16 | 69.81 ± 9.87 | 0.009 | ** |

* p < 0.05
** p < 0.01

Analysis of repeated measurement data was used to compare differences between base line (0 week) and various time intervals.

As shown in Table 11, starting from week 4, the water content of the skin increased significantly compared with that of the baseline.

TABLE 12

| Variable | Mean | Std Dev | t Value | Pr > \|t\| |
|---|---|---|---|---|
| diff1/8 | 13.6188889 | 13.3251486 | 3.07 | 0.0154 |
| diff7/8 | −2.5262500 | 9.4680107 | −0.75 | 0.4751 |

As shown in Table 12, there was significant difference between week 0 and week 16 (diff ⅛), and no significant difference between week 12 and week 16(diff ⅞).

It appeared that discontinuation of the treatment for 4 weeks, there was still delayed effect of SS304L on skin hydration.

(C) Hematology, blood chemistry and urine analysis

TABLE 13

| | |
|---|---|
| Subject number | 10 (female) |
| Age | 40-50 years old |
| Hematology (W/RBC, ESR, | There were 2 missing data, There were |
| Hemoglobin, Hematocrit, | two subjects with ESR out of normal |
| Platelet count) | range at baseline, and their values were |
| | back to normal at week 12. |
| Blood chemistry (SGOT, | There were two missing data. There was |
| SGPT, Alkaline phosphatase, | one subject who had higher values of |
| Creatinine, LDH, BUN) | SGOT and SGPT than normal at baseline |
| | and remained high at week 12; one subject |
| | starting with high LDH at baseline, and at |
| | borderline high at week 12. |
| Urine analysis (Protein, | There were 2 missing data, The |
| Glucose, W/RBC) | parameters checked were normal of all of |
| | the subjects. |

As shown in Table 13, there were no apparent side effects of SS304L on vital signs, hematology, blood chemistry and urine analysis after three months of dosing of SS304L.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A composition comprising Semen Lactucae sativae, Fructus oryzae and Resina Boswelliae carterii.

2. The composition of claim 1, wherein the Semen Lactucae sativae, Fructus oryzae and Resina Boswelliae carterii are presented at the weight to weight ratio of 2-20:1-5:0.1-1.

3. The composition of claim 2, wherein said ratio is 5:2:0.5.

4. The composition of claim 1, wherein Resina Boswelliae carterii is resin containing Resina Boswelliae carterii.

5. The composition of claim 1, which is in a form of tablets, film coated tablets, capsules, soft gel capsules, granules, powder, pearl, liquid, emulsion, injection solution, ointment, lotion, cream, serum, spray, inhaler or skin covering by employing with a pharmaceutically acceptable excipient, carrier or diluent.

6. A method of preparing the composition of claim 1 comprising:(1) extracting from Semen Lactucae sativae, Fructus oryzae and Resina Boswelliae carterii at the weight to weight ratio of 2-20:1-5:0.1-1 with 5 to 10 fold volume of water at 95 to 105° C. for 2 hours twice; and (2) mixing the effective amount of the extract with a pharmaceutically acceptable excipient, carrier or diluent to various expecting dosage forms.

7. A method for prophylaxis or treatment of skin-aging of a subject comprising administrating an effective dose of a composition to a subject, wherein the composition comprises Semen Lactucae sativae, Fructus oryzae and Resina Boswelliae carterii.

8. The method of claim 7, wherein the weight to weight ratio of said composition is 2-20:1-5:0.1-1.

9. The method of claim 8, wherein said ratio is 5:2:0.5.

10. The method of claim 7, wherein the composition is administered via oral, intra-muscular injection, subcutaneous injection, intra-venous injection, mucosal membrane or topical routes.

11. The method of claim 7, wherein said composition is in a form of tablets, film coated tablets, capsules, soft gel capsules, granules, powder, pearl, liquid, emulsion, injection solution, ointment, lotion, cream, serum, spray, inhaler or skin covering.

12. A method for facilitating wound healing rate and improving wound quality of a patient comprising administrating an effective dose of the composition of claim 1 to a patient.

13. The method of claim 12, wherein the weight to weight ratio of said composition is 2-20:1-5:0.1-1.

14. The method of claim 13, wherein said ratio is 5:2:0.5.

15. The method of claim 12, wherein said composition is administered via oral, intra-muscular injection, subcutaneous injection, intra-venous injection, mucosal membrane or topical routes.

16. The method of claim 12, wherein the composition is in a form of tablets, film coated tablets, capsules, soft gel capsules, granules, powder, pearl, liquid, emulsion, injection solution, ointment, lotion, cream, serum, spray, inhaler, skin covering or wound dressings.
